(19) United States
(12) Patent Application Publication
(10) Pub. No.: US 2023/0247947 A1
(43) Pub. Date: Aug. 10, 2023

(54) CELLULOSE NANOCRYSTALS FOR PLANT STARTER PLUGS

(71) Applicant: The Florida State University Research Foundation, Inc., Tallahassee, FL (US)

(72) Inventor: Gang Chen, Tallahassee, FL (US)

(21) Appl. No.: 18/100,813

(22) Filed: Jan. 24, 2023

Related U.S. Application Data
(60) Provisional application No. 63/308,779, filed on Feb. 10, 2022.

(51) Int. Cl. A01G 24/22; B82Y 5/00; C12N 9/42

(52) U.S. Cl. CPC A01G 24/22; B82Y 5/00; C12N 9/2437; C12Y 302/01004

(57) ABSTRACT

The present disclosure provides for a method of making a plant growth medium comprising isolating cellulose nanocrystals from a feedstock, and adding the cellulose nanocrystals to the plant growth medium. Further provided herein is a plant starter plug comprising the plant growth medium. Also provided herein is a plant growth medium comprising germination material and cellulose nanocrystals, and a plant starter plug comprising thereof. The present disclosure further provides for a method of isolating CNCs from waste.

### CROSS REFERENCE TO RELATED APPLICATIONS

This application claims the benefit of priority to U.S. Provisional Application No. 63/308,779, filed Feb. 10, 2022, the disclosure of which is incorporated herein by reference in its entirety.

### BACKGROUND

As the human population around the world continues to grow and concerns regarding climate change continue to grow, “urban farming” has become a prevalent alternative to conventional farming. The urban farming market had an estimated market value of $213 billion in 2020 and is projected to grow 2.8% from 2021 to 2026. Crops grown via urban farming may include non-food crops, such as medicinal herbs, ornamental plants, and aromatic herbs, as well as food crops, such as fruits, vegetables, grains, and cereals. Urban farming methods include aeroponics, hydroponics, and aquaponics. Aeroponics involves plant growing while suspended in air, hydroponics involves plant growing while suspended in water, and aquaponics is the combination of hydroponics and fish farming in an integrated system.

Benefits of urban farming include the ability to supply a significant number of city dwellers with diverse and secured food, thus promoting agricultural diversity and food security. Additionally, urban farming fosters less transportation and therefore less greenhouse gas emissions. Urban farming also promotes organic farming.

Plant starter plugs are often used for seed germination during urban farming. As the urban farming market continues to grow, the market for plant starter plugs continues to grow as well. The plant starter plugs in use presently need to be watered regularly in order to maintain the moisture content required for effective seed germination. When the plants are not watered regularly, the seeds are unable to germinate and grow, thus causing a low crop yield. Thus, there is a need for a product and method that lessen or eliminate the need to regularly water the seeds in the plant starter plugs, for example by enhancing the ability of the plant starter plugs to maintain necessary water levels. Further, there is a need for a product and method that achieves the goals with limited chemical use. The compositions and methods disclosed herein address these and other needs.

### SUMMARY

In accordance with the purposes of the disclosed materials and methods, as embodied and broadly described herein, the disclosed subject matter, in one aspect, relates to compositions and methods of making compositions. In specific aspects, the disclosed subject matter relates to a plant growth medium and a method of making thereof.

Thus, in one example, a plant growth medium is provided including germination material and cellulose nanocrystals.

In a further example, methods of making a plant growth medium are provided including isolating cellulose nanocrystals from feedstock and adding the cellulose nanocrystals to the plant growth medium.

In some examples, a method is provided for obtaining cellulose nanocrystals, including treating cellulosic waste paper feedstock sample with base, washing and drying the sample, bleaching the sample by contacting it with sodium hypochlorite, hydrolyzing the sample by contacting it with sulfuric acid, and washing and dialyzing the sample to obtain the cellulose nanocrystals.

Additional advantages will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### DESCRIPTION OF THE DRAWINGS

The accompanying figures, which are incorporated in and constitute a part of this specification, illustrate several aspects described below.

FIG. 1 is a graph of the equilibrium moisture content percentage of an exemplary CNC as a function of relative humidity.

FIG. 2 shows an exemplary aeroponic system.

FIG. 3 shows an exemplary hydroponic system.

FIG. 4 shows an exemplary aquaponic system.

FIG. 5 shows example OH surface groups that can provide active sites for hydrogen bonding through the interlocking with a nonpolar matrix.

FIG. 6 is a graph of crop water uptake with respect to soil moisture and soil moisture potential.

FIG. 7 shows the components of a fibril from the cell wall of exemplary feedstock.

FIG. 8 is a schematic demonstrating CNC extraction from cellulose.

FIG. 9 is a schematic of a cell wall structure from exemplary feedstock.

FIG. 10 is a schematic of feedstock comprising lignin, cellulose, and hemicellulose.

FIG. 11 is a schematic of an exemplary dialysis technique as used in acid extraction.

FIG. 12 is a diagram of different exemplary CNC dimensions and crystal ratios.

FIG. 13 is an image of an exemplary ultrasonic device.

FIG. 14 is a schematic of the high energy bead milling process.

FIG. 15 is a schematic of the result of pretreatment of feedstock with cryocrushing.

FIG. 16 shows an example CNC extraction process.

FIGS. 17A-17C show FTIR spectra of the raw material and extracted CNC.

FIGS. 18A-18C show TEM micrographs of the obtained CNC from waste corrugated cardboards (WCC), wastepaper towels (WPT), and wastepaper towel cardboard rolls (WPTR).

FIG. 19 shows the distribution of the diameter of the obtained CNC from WCC, WPT, and WPTR.

FIGS. 20A-20C shows XRD analysis of the obtained CNC from WCC, WPT, and WPTR.

### DETAILED DESCRIPTION

The following description of the disclosure is provided as an enabling teaching of the disclosure in its best, currently known embodiments. Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As can be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It can be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.”

As used in the specification and the appended claims, the singular forms “a,” “an” and “the” include plural referents unless the context clearly dictates otherwise. Thus, for example, reference to “a compound”, “a composition”, or “a disorder”, includes, but is not limited to, two or more such compounds, compositions, or disorders, and the like.

When a range is expressed, a further aspect includes from the one particular value and/or to the other particular value. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

### Methods

### Methods of Making Plant Growth Medium

The present disclosure provides for a method of making a plant growth medium, including isolating cellulose nanocrystals from feedstock; and adding the cellulose nanocrystals to the plant growth medium. “Cellulose nanocrystal (CNC)” is used herein to refer to a cellulosic object that includes the predominately crystalline regions extracted from cellulose, wherein those regions do not exhibit branches or entanglement between the CNCs or network-like structures. This differs from cellulose nanofibers and nanofibrils (CNFs), as they contain crystalline as well as amorphous regions with entanglement between cellulose nanofibers or nanofibrils, respectively. The extraction of CNFs can be through mechanical or a combination of mechanical and chemical methods (Kaushik, Singh, & Verma, 2010).

Wood is one material for nanocellulose (NC) extraction because of its abundance and high percentage of cellulose content (Fernandes, Pires, Mano, & Reis, 2013). Other materials such as rice husk (Johar, Ahmad, & Dufresne, 2012), sisal (Mariano, Cercená, & Soldi, 2016), cotton (Pandi, Sonawane, & Anand Kishore, 2021), tunicate (Sacui et al., 2014), coconut husk (M. F. Rosa et al., 2010), and algae (El Achaby, Kassab, Aboulkas, Gaillard, & Barakat, 2018) are also good sources of cellulose.

In some embodiments, cellulose nanocrystals can be cylindrical, elongated, less flexible, rod-like, or any combination thereof. In certain embodiments, CNCs can include only cellulose, while in other embodiments, they can also include lignin, hemicellulose, or any combination thereof. In some embodiments, CNCs have high crystallinity degree, large specific surface area, high thermal resistance, high mechanical properties, biodegradability, and/or biocompatibility.

In some embodiments, isolating cellulose nanocrystals can include extracting cellulose from feedstock and extracting the cellulose nanocrystals from the cellulose. “Cellulose” is used herein to refer to the primary structural component of plant cell walls, consisting of long cellobiose unbranched chains of d-anhydroglucose units with β-(1,4) glycosidic bonds. Cellulose has both crystalline and amorphous domains with different interactions between the oxygen atoms and hydroxyl groups in each d-anhydroglucose unit within a linear polymeric chain or with other polymeric chains that generate the secondary valence bonds and contribute to the crystalline structure of cellulose (Sasaki, Adschiri, & Arai, 2003).

In some embodiments, extracting cellulose from the feedstock can include acidic or enzymatic hydrolysis. “Hydrolysis” is used herein to refer to the chemical reaction in which long-chain compounds are broken into shorter-chain substances. In some embodiments, hydrolysis can break down feedstock so that hemicellulose and lignin can be separated from cellulose, while in other embodiments hydrolysis can break down cellulose so as to extract cellulose nanocrystals.

In some embodiments, acidic hydrolysis includes, but is not limited to, hydrolyzing the feedstock with hydrochloric acid (HCl). In other embodiments, acid hydrolysis can include hydrolyzing cellulose with sulfuric acid (H₂SO₄). In further embodiments, the acids used for acid hydrolysis can include, but are not limited to, trifluoroacetic acid (CF₃COOH), formic acid (HCOOH), and nitric acid (HNO₃).

In some embodiments, enzymatic hydrolysis can be used to hydrolyze cellulose instead of acid hydrolysis. “Enzymatic hydrolysis” is used herein to refer to the biochemical reaction in which cell wall components are broken down by enzymes. In some embodiments, enzymes include cellulose enzymes, hemicellulose enzymes, or a combination thereof. Cellulose enzymes can include, but are not limited to, cellulase and more specifically, endo-1,4-β-D-glucanase, exo-1,4-β-D-glucanase and β-glucosidase. In further embodiments, cellulose enzymes can include cellulolytic enzymes from Trichoderma reesei. Hemicellulose enzymes include, but are not limited to, endoxylanase, beta-xylosidase, alpha-L-arabinofuranosidase, alpha-glucurondiase, alpha-galatosidase, acetylxylan esterase, feruloyl esterase, or any combination thereof.

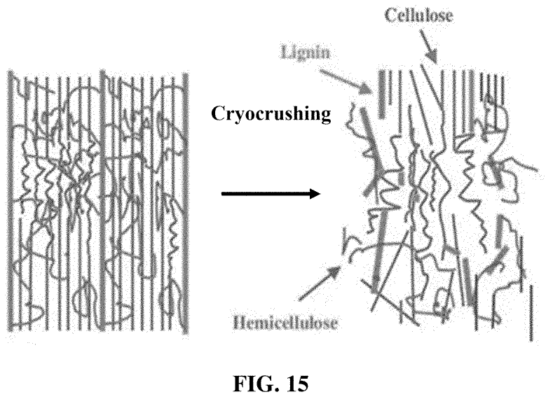

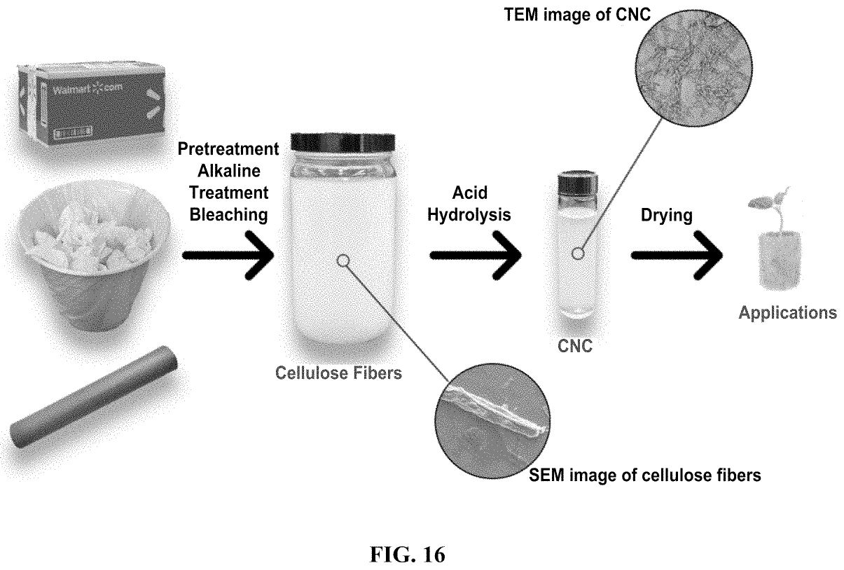

In further embodiments, extracting cellulose from the feedstock can include cryocrushing the feedstock. “Cryocrushing” is used herein to refer to the process of freezing material using liquid nitrogen and subsequently applying high impact forces to the frozen material. In some embodiments, the frozen material can be feedstock and the application of high impact forces separate fibrils, such as lignin and hemicellulose from cellulose. (FIG. 15) In certain embodiments, feedstock is soaked in liquid nitrogen for 24 hours to make the cellulosic materials more brittle and is then crushed using mortar and pestle.

In particular embodiments, extracting cellulose from the feedstock can further include high shear grinding. “High shear grinding” is used herein to refer to a high shear dispersion and/or dissolution process. High shear grinding can include, but is not limited to, batch or inline designs, multi-agitator systems, ultra-high shear rotor/stator geometries, or any combination thereof.

In some embodiments, extracting cellulose nanocrystals from the cellulose can include acidic or enzymatic hydrolysis. Acidic hydrolysis and enzymatic hydrolysis are used as described herein. In certain embodiments, enzymatic hydrolysis can use cellulolytic enzymes from Trichoderma reesei. In other embodiments, cellulolytic enzymes can include fungi belonging to the genera Penicillium, Acremonium, Chrysosporium, or any combination thereof.

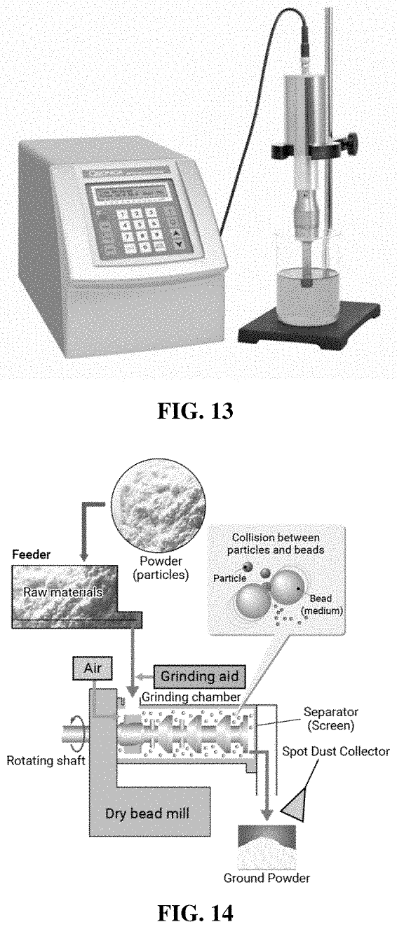

In further embodiments, extracting cellulose nanocrystals can include performing ultrasonication, high energy bead milling, or any combination thereof on the cellulose. “Ultrasonication” is used herein to refer to the irradiation of a liquid sample with ultrasonic waves to cause agitation. The device used for ultrasonication can include an ultrasonic processor with programmable operations, such as, for example, the Q500 Sonicator (manufactured by QSonica, Newtown, Conn.). (FIG. 13) In some embodiments, the ultrasonic waves have a frequency greater than 20 kHz. In further embodiments, cellulose is ultrasonicated for 50 minutes at a frequency of 20 kHz.

“High energy bead milling” is used herein to refer to the bead milling process in which a powder mixture is placed in a bead mill and subjected to high-energy collisions from the beads. (FIG. 14) The bead mills used for high energy bead milling are machines for dispersion processing and can include, but are not limited to, small ceramic, glass, or metal beads that are agitated inside the mill chamber to aid particle size reduction through impact and energy input. Depending on the design of the bead mill, high energy bead milling can occur as a batch process or continuous process. In some embodiments, high energy bead milling can include using an agitator bead mill with 0.4 mm zirconium beads in a batch process mode at 1000 rpm for 15, 20, or 60 minutes.

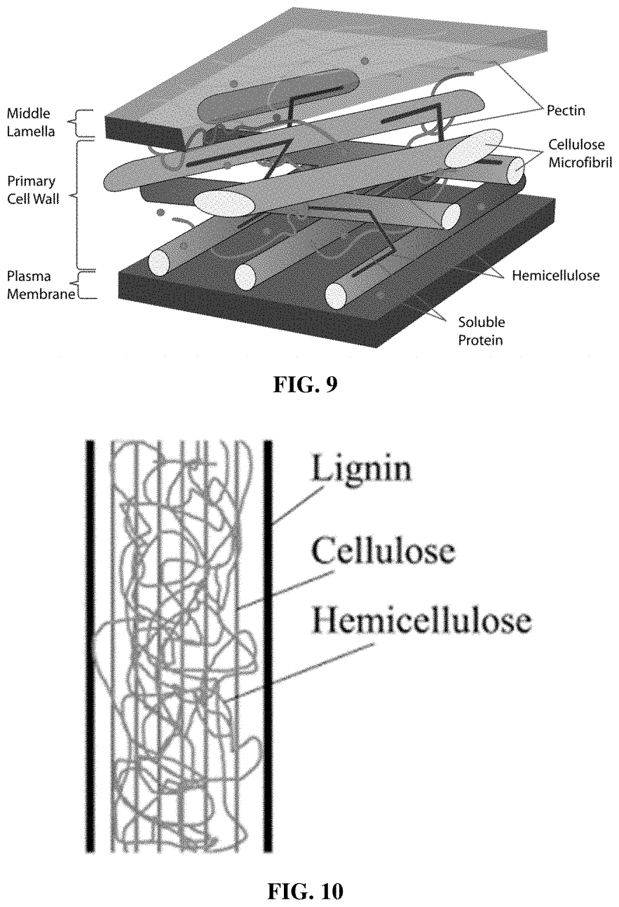

In some embodiments, the feedstock can include coconut husk fiber, sugarcane bagasse, wheat straw, corn stover, coconut shell, cotton, rice hulls, or any combination thereof. “Feedstock” is used herein to refer to the materials containing cellulose. In some embodiments, feedstock can include, but is not limited to, crop residues or wood residues, like wheat straw or corn stover, having a makeup of cellulose, hemicellulose, lignin, and/or pectin. In some embodiments, feedstock comprises 35-65 wt. % cellulose, 20-45 wt. % hemicellulose, and 10-25 wt. % lignin. (FIG. 10) In other embodiments, feedstock can include renewable organic material that comes from plants or animals. In further embodiments, feedstock can include other sources of cellulose, including but not limited, cotton, coconut husk fiber, sugarcane bagasse, wheat straw, corn stover, coconut shell, cotton, rice hulls, or any combination thereof.

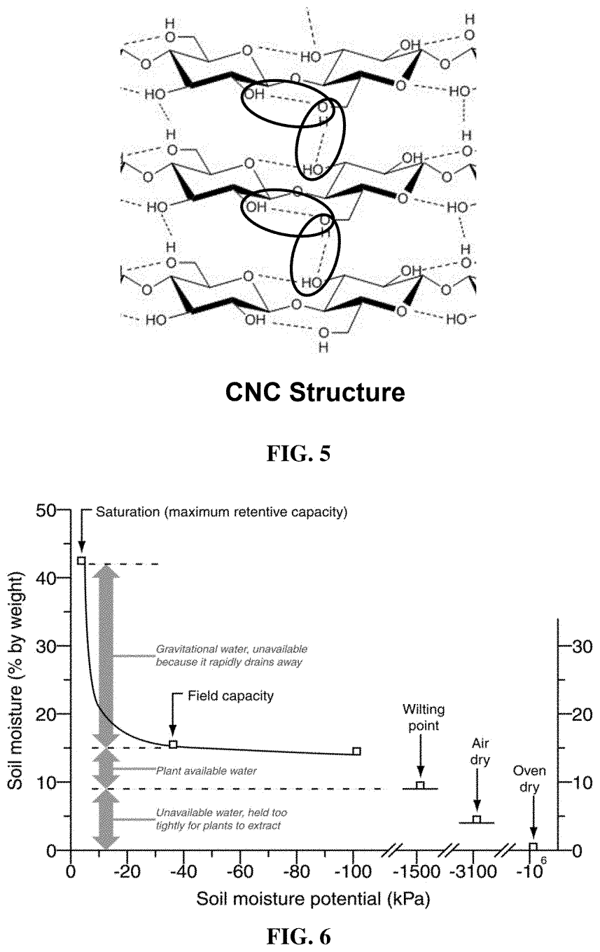

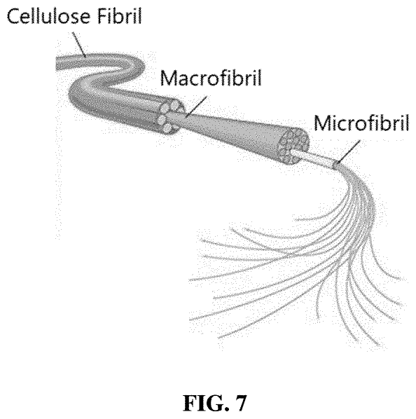

In some embodiments, the plant growth medium can maintain an appropriate moisture level without irrigation. “Appropriate moisture level” is used herein to refer to the amount of water that is in the soil or material surrounding a specimen such that there is water available for the specimen to germinate and grow (FIG. 6). The soil moisture % by weight at which water is available to the specimen corresponds to a soil moisture potential between field capacity and wilting point. In some embodiments, field capacity corresponds to a soil moisture potential of −40 kPa and wilting point corresponds to a soil moisture potential of −1500 kPa (FIG. 6).

“Irrigation” is used herein to refer to supplying water to a material, plant, or seed, for example, by means which include, but are not limited to, an irrigation system, sprinkler system, or watering can. In some embodiments, irrigation requires human involvement, whether in the form of physically providing water or programming an irrigation or sprinkler system to supply water, for example. In further embodiments, irrigation includes regularly supplying water, such as on a daily or weekly basis, for example.

In certain embodiments, the cellulose nanocrystal can extract water vapor from surrounding air. In other embodiments, the cellulose nanocrystal can retain water vapor. “Water vapor” as used herein refers to the gaseous phase water within the hydrosphere. Water vapor makes up 99% of atmospheric water and is generated by evaporation and collected by condensation.

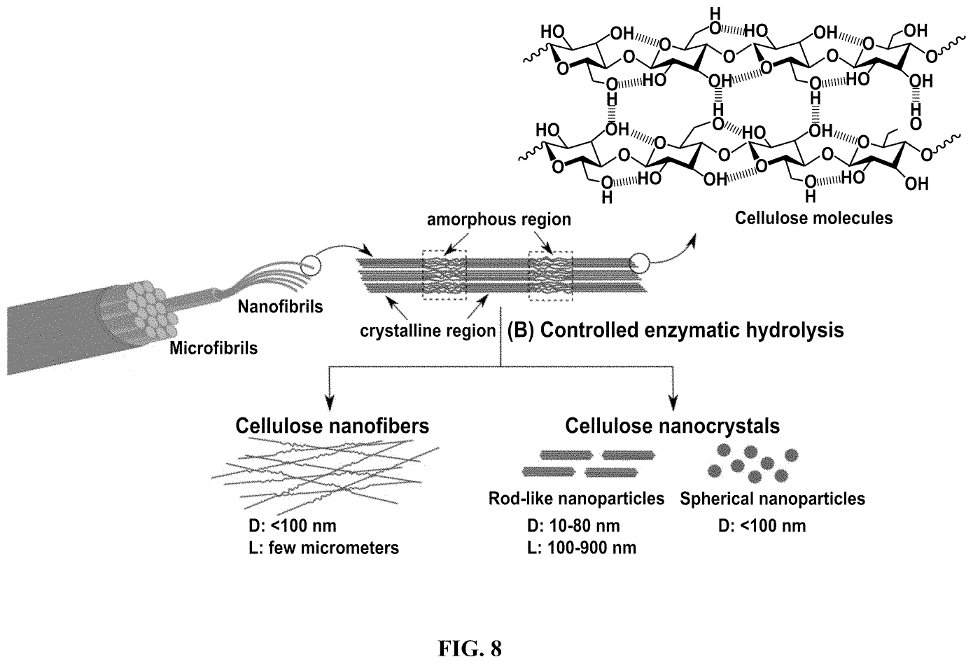

In some embodiments, the cellulose nanocrystals can be rod-shaped (FIG. 8). In other embodiments, the cellulose nanocrystals can be spherical (FIG. 8).

In some embodiments, the cellulose nanocrystals do not include lignin or hemicellulose (FIG. 10).

In some embodiments, the cellulose nanocrystals can have a width from 3 nm to 100 nm. In other embodiments, CNCs can have a width from 7 nm to 50 nm or from 50 nm to 100 nm. In further embodiments, CNCs can have a width from 10 nm to 80 nm, 10 nm to 30 nm, 30 nm to 50 nm, 50 nm to 65 nm, and 65 nm to 80 nm.

In some embodiments, the cellulose nanocrystals can have a length from 50 nm to 6 μm. In other embodiments, CNCs can have a length from 75 nm to 750 nm or from 750 nm to 2 μm. In further embodiments, CNCs can have a length from 100 nm to 900 nm, 100 nm to 300 nm, 300 nm to 500 nm, 500 nm to 700 nm, or 700 nm to 900 nm.

In some embodiments, the cellulose nanocrystals can have a crystal aspect ratio from 10 to 200. “Crystal aspect ratio” is used herein to refer to the dimension of the major axis of the crystal divided by the dimension of the minor axis. In some embodiments, crystal aspect ratio can be the width of a nanocrystal divided by the length of the nanocrystal. In some embodiments, CNCs can have a crystal aspect ratio, calculated by dividing length (major axis) by width (minor axis), from 10 to 200. In other embodiments, CNCs can have a crystal aspect ratio from 20 to 100. In further embodiments, CNCs can have a crystal aspect ratio from 30 to 70, 30 to 40, 40 to 50, 50 to 60, or 60 to 70.

In some embodiments, the plant growth medium can include a plant seed. “Plant seed” is used herein to refer to a plant's unit of reproduction, wherein the seed develops into another such plant. Plant seeds can include seeds for non-food crops such as medicinal herbs, ornamental plants, or aromatic herbs, as well as food crops, such as fruits, vegetables, grains, or cereals, for example.

In some embodiments, the plant growth medium can include a germination material. In further embodiments, the germination material can include soil, coco peat, rockwool, coco coir, or any other combination thereof. “Germination material” is used herein to refer to the material in which seeds are placed for germination in crop growing methods such as aquaponics, hydroponics, or aeroponics. In some embodiments, germination material can include, but is not limited to potting or gardening soil, coco peat, rockwool, coco coir, or any combination thereof. In other embodiments, germination material can include perlite, sand, rock salt, oasis cubes, sponges, clay pebbles, rocks, gravel, sandstone, growstone, vermiculite, rice hulls, sawdust, peat moss, or any combination thereof.

In some embodiments, disclosed herein is a plant starter plug comprising any one of the plant growth mediums made by the methods discussed herein. “Plant starter plug” is used herein to refer to a small, compact mass or sample of a solid growing medium that is used for seed germination, with a sponge-like consistency that protects root structures of young plants to ensure safe transplanting into soil or soil-less growth media. In some embodiments, plant starter plugs can keep consistent moisture levels, provide aeration, and lack disease. In certain embodiments, materials used in plant starter plugs include, but are not limited to, coco peat, rockwool, coco coir, or any combination thereof. Coco peat is a byproduct of coco production having loose particles to be washed around the system, a pH between 5.0 and 6.8, and the ability to enhance the oxygenation process. Rockwool is a silica-based material that can be heated and spun into thin threads having an ideal oxygen to water ratio and a pH between 7.8 and 7.9. Coco coir is coconut hair and, in some embodiments, sediment on the coconut hair is washed off so that it does not clog any pumps utilized in the plant-growing system.

### Composition

### Plant Growth Medium

The present disclosure provides a plant growth medium including germination material and cellulose nanocrystals. “Germination material”, and “cellulose nanocrystal” are used as described herein. While not wishing to be bound by theory, the plant growth medium sustains seed germination without irrigation because the cellulose nanocrystals can extract water vapor from the air.

In some examples, the plant growth medium can include a plant seed, wherein “plant seed” is used as described herein.

In some examples, the cellulose nanocrystals in the plant growth medium can be substantially free of amorphous cellulose. “Amorphous cellulose” is used herein to refer to the portions of cellulosic materials that are solid but exhibit no crystalline structure. Cellulose nanofibrils and cellulose nanofibers, for example, can contain amorphous cellulose. (FIG. 8) In some examples, cellulose nanocrystals do not contain amorphous cellulose. In further examples, the plant growth medium can include isolated cellulose nanocrystals. Further, in certain examples the plant growth medium can include concentrated cellulose nanocrystals.

In some embodiments the germination material can include soil, coco peat, rockwool, coco coir, or any combination thereof. Further embodiments of germination material are described herein.

In some embodiments, the plant growth medium can maintain an appropriate moisture level without irrigation. Moisture level as used is described herein.

In certain embodiments, the cellulose nanocrystal can extract water vapor from surrounding air. In other embodiments, the cellulose nanocrystal can retain water vapor. Water vapor is used as described herein.

In some embodiments, the cellulose nanocrystals can be rod-shaped (FIG. 8). In other embodiments, the cellulose nanocrystals can be spherical (FIG. 8).

In some embodiments, the cellulose nanocrystals do not include lignin or hemicellulose (FIG. 10).

In some embodiments, the cellulose nanocrystals can have a width from 3 nm to 100 nm. In other embodiments, CNCs can have a width from 7 nm to 50 nm or from 50 nm to 100 nm. In further embodiments, CNCs can have a width from 10 nm to 80 nm, 10 nm to 30 nm, 30 nm to 50 nm, 50 nm to 65 nm, and 65 nm to 80 nm.

In some embodiments, the cellulose nanocrystals can have a length from 50 nm to 6 μm. In other embodiments, CNCs can have a length from 75 nm to 750 nm or from 750 nm to 2 μm. In further embodiments, CNCs can have a length from 100 nm to 900 nm, 100 nm to 300 nm, 300 nm to 500 nm, 500 nm to 700 nm, or 700 nm to 900 nm.

In some embodiments, the cellulose nanocrystals can have a crystal aspect ratio from 10 to 200. Crystal aspect ratio is used as described herein. In other embodiments, CNCs can have a crystal aspect ratio from 30 to 70, 30 to 40, 40 to 50, 50 to 60, or 60 to 70.

In some embodiments, disclosed herein is a plant starter plug comprising any one of the plant growth mediums discussed herein. Plant starter plug is used as described herein.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

### Example 1: Cellulose Extraction Method

Cotton, coconut shell and rice hulls were used for cellulose nanocrystal extraction. In the pretreatment, hemicellulose and lignin were removed from the feedstock in the following steps: 1) the fiber was hydrolyzed for four hours with hydrochloric acid (2.5N) at 80° C. to solubilize the hemicellulose material; 2) the resulting product was washed with DI water three times; 3) the product was then waterlogged in NaOH (5 wt. %) for two hours to make it susceptible to hydrolysis by increasing the surface area of the cellulosic material; and 4) the product was treated with NaClO at 75° C. to interrupt the lignin structure. This process was performed two times. The overall mass yield of produced cellulose was 10-20% of the feedstock.

### Example 2: Using Sodium Hydroxide to Remove Hemicellulose and Lignin

The feedstock made of straw and stover natural fibers was waterlogged with NaOH (15 wt. %) for 2 hours to make it susceptible to hydrolysis by increasing the surface area of the cellulosic materials. Next, the fibers were hydrolyzed with hydrochloric acid (HCl) at 75° C. to solubilize the hemicellulose materials. The fibers then underwent an alkaline treatment with NaOH (2 wt. %) at 75° C. to interrupt the lignin structures.

### Example 3: Cellulose Extraction by Cryocrushing

Feedstock was soaked in liquid nitrogen for twenty-four hours to make feedstock more brittle. Brittle feedstock was then cryocrushed via strenuous crushing with a mortar and pestle. To achieve uniformity of the cellulose fibrils, cryocrushing was combined with high shear grinding. This method avoids overheating, which can be an issue with high-shear grinding alone, because of the use of liquid nitrogen. The use of cryocrushing and high-shear grinding instead of acid hydrolysis allowed for the use of less chemicals in the cellulose extraction process.

### Example 4: Cellulose Nanocrystal Extraction Method

Cellulose nanocrystals were produced using an extracted cellulose solution. The cellulose solution was hydrolyzed for two hours with sulfuric acid (H₂SO₄) (60 wt. %). The solution was mechanically stirred during the hydrolysis reaction and washed with cold de-ionized water. The solution was then centrifuged three times with a 0.45 μm filter to remove any residuals after the acid hydrolysis.

### Example 5: Cellulose Nanocrystal Acid Extraction

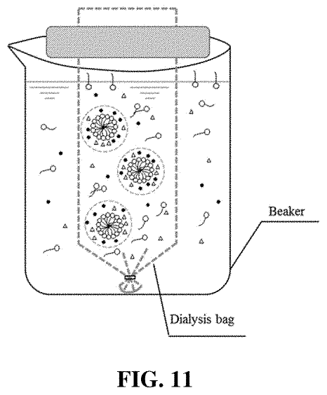

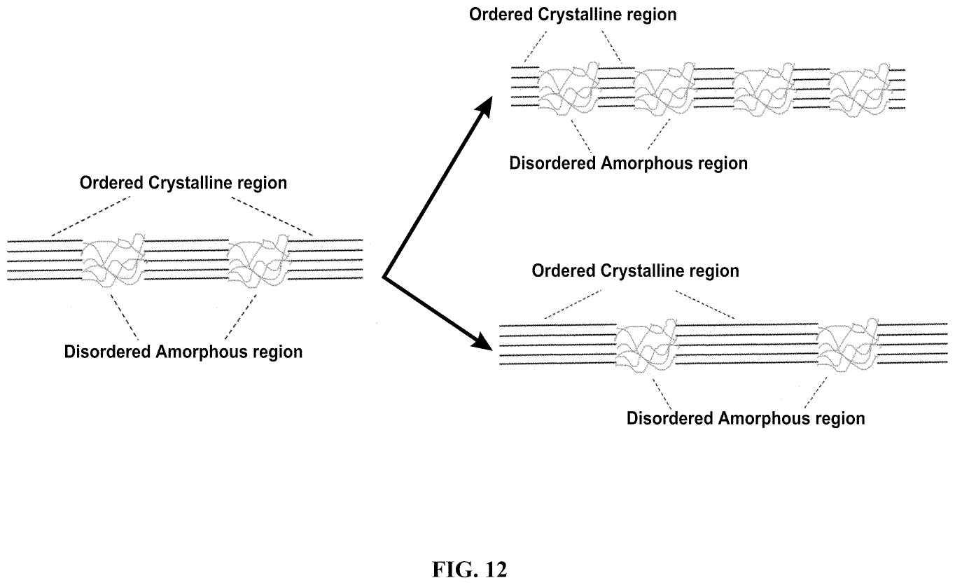

Cellulose was hydrolyzed with 60 wt. % H₂SO₄ at 55° C. for 102 minutes and then mechanically stirred. Cold deionized water was then added, and the solution washed three times via centrifuge at 16,000×g for 10 minutes. The solution was then dialyzed against deionized water to a constant pH. (FIG. 11) Next, the cellulose was sonicated in an ice-water bath for 30 minutes and then centrifuged at 9000×g for 5 minutes. Lastly, the resulting solution underwent supernatant spray drying using a laboratory scale dryer (e.g., Mini Spray Dryer B-290, manufactured by BÜCHI Labortechnik AG, Flawil, Switzerland).

### Example 6: Cellulose Nanocrystal Enzymatic Extraction

Cellulolytic enzymes from the microscopic fungus Trichoderma reesei were added to the cellulose chain with dilution by 1:50 citrate buffer. The sample was incubated at 50° C. for 24 hours with a shaking speed of 250 rpm/minute. The sample was then dialyzed against de-ionized water to a constant pH. Next, the sample was sonicated in an ice-water bath for 30 minutes and centrifuged at 9000×g for five minutes. The sample then underwent supernatant spray-drying using a laboratory-scale dryer (i.e., Mini Spray Dryer B-290, manufactured by BÜCHI Labortechnik AG, Flawil, Switzerland). The dimensions and crystal ratios of the cellulose nanocrystals were impacted by the enzyme load, temperature at which enzyme hydrolysis is performed, and incubation time, which in turn enhanced the water vapor extraction capabilities of the cellulose nanocrystals.

### Example 7: Moisture Content of Cellulose Nanocrystals

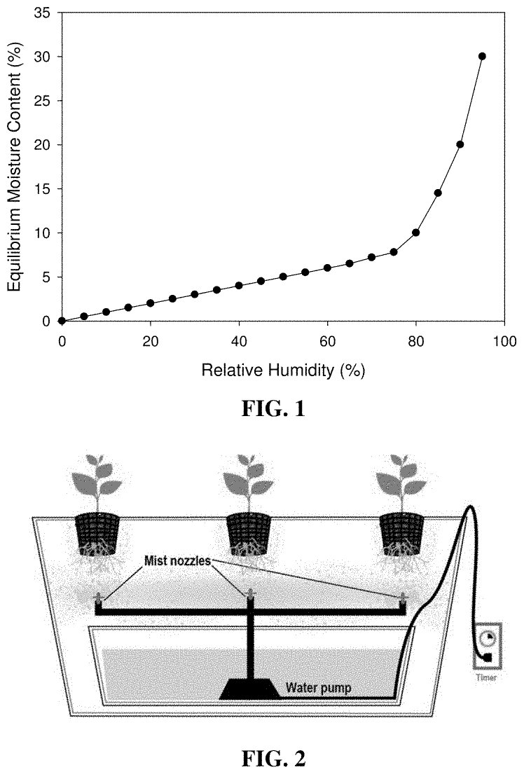

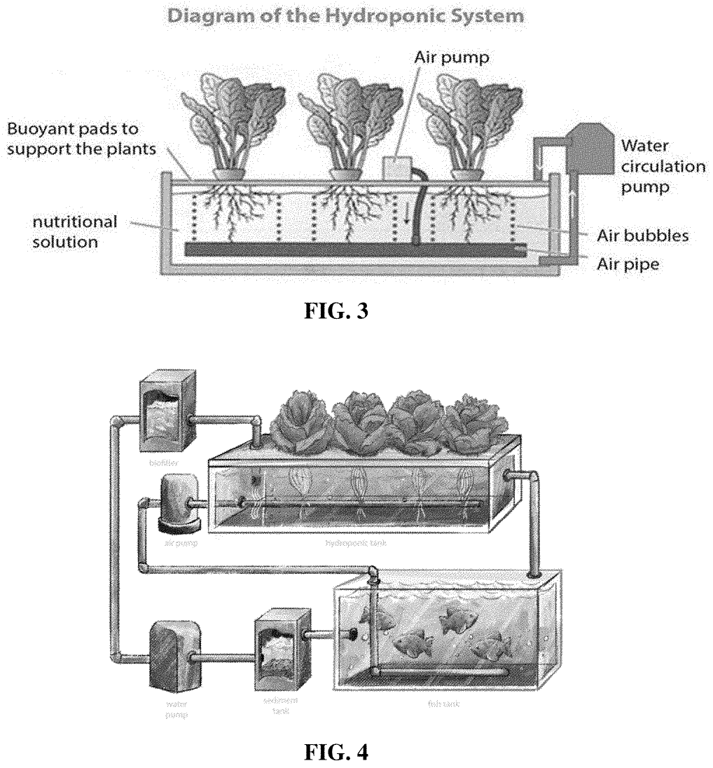

Cellulose nanocrystals (CNCs) were isolated from agricultural waste by chemical induction via removing the amorphous region. The chemical treatment, mechanical treatment, or combination involved enzymatic treatment, grinding, high-pressurized homogenization, acid hydrolysis, TEMPO-mediated oxidation, micro-fluidization, cryocrushing, and high-intensity ultrasonication. In the lab, sulfuric acid was used to hydrolyze the feedstocks of coconut husk fiber and sugarcane bagasse to produce CNCs with negatively charged sulfate groups. The acid was then disposed of following a dialysis process. The concentration of acid, acid-to-fiber ratio, temperature, and time of the hydrolysis process were controlled to produce CNCs with different dimensions and crystal ratios. The moisture extraction capacity of the CNCs was tested in the lab. It was determined that the water vapor extraction was a function of the relative humidity (FIG. 1). In practice, CNCs can extract abundant moisture for the plant starter plugs at high relative humidity and at low humidity, the CNCs can retain the water to prevent water loss.

### Example 8: CNC Extraction by Ultrasonication and High-Energy Bead Milling

Ultrasonication was performed for 50 minutes at a frequency of 20 kHz. After separation, the top layer was freeze-dried. High-energy bead milling was performed by using an agitator bead mill with 0.4 mm zirconium beads in a batch process mode at 1000 rpm for 15, 30, or 60 minutes. The dispersion was then freeze-dried. The dimensions and crystal ratios of the cellulose nanocrystals were impacted by the ultrasonication time, frequency and power at which ultrasonication were performed, the bead milling rpm, and the length of time of high-energy bead milling, which in turn enhanced the water vapor extraction capabilities of the cellulose nanocrystals. The use of ultrasonication and high-energy bead milling instead of acid hydrolysis allowed for the use of less chemicals in the CNC extraction process.

### Example 9: Characterization of Cellulose Nanocrystals Extracted from Household Wastes

Cellulose nanocrystals (CNCs) were extracted and characterized from three different types of household wastes including waste corrugated cardboard (WCC), wastepaper towels (WPT), and wastepaper towel cardboard rolls (WPTR) in this research. Instead of being deposited into landfills, these materials were found to have environmental benefits to serve as a low-cost and readily available precursor to the production of cellulose nanomaterials. A modified acid hydrolysis method was used in this study for the isolation of CNCs, which exhibited more environmentally friendly features. Fourier transform infrared spectroscopy (FTIR) and X-ray diffraction (XRD) were used for the characterization of CNC composition and structure and the morphology of isolated cellulose and CNCs was examined using field emission scanning electron microscopy (FESEM) and transmission electron microscopy (TEM), respectively. The CNCs extracted from the three feedstocks were needle/whisker-shaped with sizes ranging from 4 to 15 nm. The CNCs extracted from WPT had a smaller size in comparison with the two other samples. The crystallinity of the CNCs extracted from WCC, WPT, and WPTR was 78.1%, 83.3%, and 77.5%, respectively.

Resource recovery from the municipal solid waste has been a focus of solid waste management. Cellulose, as a biodegradable material, can be potentially used for finite purposes such as papermaking, clothing, pharmaceuticals, etc. (Mohanty, Misra, & Drzal, 2002).

Nanocellulose (NC) has properties such as exceptional mechanical strength, large specific surface area, high aspect ratio, low thermal expansion, high Young's modulus, and so on (Chirayil et al., 2014; Wei, Rodriguez, Renneckar, & Vikesland, 2014).

Paper pulp, which usually comes from wood, is the main raw material in almost all paper-based products and is rich in cellulose. As a cellulose biomass, paper-based products and wastes can be feedstock for CNC and CNF extraction, particularly given the fact that they are derived from wood and at the same time lack the stiffness and toughness of wood, and therefore need less rigorous chemical and/or mechanical processes for breaking them down. The use of paper-based household wastes for extracting different types of cellulose is a more sustainable and economical way in comparison to exploiting natural resources like trees. On the other hand, it should be noted that producing NC from paper-based wastes can be a substantial alternative for recycling paper material wastes, because some paper-based products such as paper tissues, towels, and napkins usually cannot be recycled and end up in landfills, leading to the production of methane during their slow decomposition. Other paper-based products such as paper containers and packaging boxes cannot be efficiently recycled, with a recycling rate of 20.8%. There were 3.8 million tons of paper tissue and towel waste produced in the United States in 2018 (EPA, 2021, 2022). In addition, the quality of recycled paper-based products is not as desirable as those that are made from virgin pulps (Bjärestrand & Alfthan, 2020).

Herein, a modified chemical extraction method was studied to isolate NC from three different paper-based household wastes including cardboard boxes, paper towels, and paper towel cardboard rolls. The cellulose content in waste corrugated cardboard and paper towel is reported to be 52.0% and 78.1%, respectively (Dutta et al., 2020; N. Sun, Rodriguez, Rahman, & Rogers, 2011). For the extraction of cellulose, we carried out alkali and bleaching treatments to remove lignin and hemicellulose, divide cellulose fibers into smaller sections, and eliminate impurities and contaminations like grease and ink. CNCs were then extracted by removing the amorphous regions of cellulose by sulfuric acid hydrolysis (Bajpai, 2014; Habibi et al., 2010; Jiang & Ma, 2000; Ng et al., 2015). The physical and chemical characterizations of the extracted CNCs and CNFs were analyzed by ATR FTIR spectroscopy, X-ray diffraction (XRD), scanning electron microscopy (SEM), transmission electron microscopy (TEM), and Thermogravimetric Analyzer (TGA).

### Materials and Methods

The cellulose sources were waste corrugated cardboards (WCC), wastepaper towels (WPT), and wastepaper towel cardboard rolls (WPTR). The WCC was shipping packages. The WPT were collected from a university dining hall, rest area, and a garbage can in a household kitchen. The WPTR was obtained from household wastes. Sodium hydroxide, sulfuric acid (95-97 percent), and sodium hypochlorite were obtained from VWR. All the chemicals were reagent grade and were used as received.

### Preparation of Cellulose

All three feedstocks were cut into 2 cm×2 cm pieces and boiled for 4 hours in distilled water. The samples were then washed several times, dried for 24 hours at 40° C. in an oven, and then grounded in a grinder. The grounded samples were treated with 5% sodium hydroxide for 4 h at 75° C. under vigorous mechanical stirring. The alkali-treated samples were washed with distilled water and filtered until a neutral pH was achieved, and then dried at 40° C. for 24 h in an oven. The bleaching process was carried out by soaking the samples in sodium hypochlorite (5% wt) under mechanical stirring for 24 h following by washing with distilled water. The bleaching step was repeated until the color of the samples turned into pure white. The white cellulose fibers were rinsed and filtered multiple times until achieving a neutral pH.

### Cellulose Nanocrystal Extraction

CNC was extracted from the cellulose samples by sulfuric acid hydrolysis using the method described before with appropriate modification (Sheltami, Abdullah, Ahmad, Dufresne, & Kargarzadeh, 2012). The hydrolysis was carried out by mixing the cellulose fibers in a solution containing 64 wt. % H₂SO₄ at 50° C. with vigorous and continual stirring for 120 min, which was determined to be the optimum time based on the findings of this study. The volumetric ratio of cellulose solution to sulfuric acid was 1:1 which found to be the optimum ratio. The reaction was stopped by adding 10-fold cold distilled water into the mixture, which was then centrifuged for 60 min at 3500 rpm to separate large particles. After removing the large cellulose fibers, the suspension was centrifuged for 45 min at 45,000 rpm and washed three times to remove excess sulfuric acid. The turbid suspension collected was subjected to dialysis against distilled water for several days until a constant pH was obtained. The resulting suspension was then sonicated for 30 min before further analysis.

### CNC Characterization

FTIR

Functional groups of the feedstocks and the derived CNCs were analyzed using Fourier transform infrared spectroscopy. The extracted CNCs were dried to form thick films before analysis. The analysis was conducted using a JASCO 6800 spectrometer equipped with single-reflection, diamond ATR accessory with 45 degree angle of incidence. The spectra were analyzed in transmittance mode between 500 cm⁻¹ and 4000 cm⁻¹, with a resolution of 4 cm⁻¹ and a total of 80 scans for each sample.

Imaging Analysis

To access the CNCs' shape and dimensions, a TEM analysis was performed using Hitachi HT7800. 10 uL of each sample was dropped on 400 mesh copper grids, coated with carbon film, for 30-60 seconds. Immediately following, each grid was negative stained with 10 uL of 2% uranyl acetate for 60 seconds. Grids were left to dry overnight before being imaged. The Image J software was used to manipulate the data to obtain CNCs' diameter.

XRD

Powder X-ray diffraction patterns were carried on Rigaku SmartLab equipped with a copper rotating anode-type generator (Cu-Ka, λ, =0.154 nm, 40 kV, 44 mA) and D/teX Ultra detector. The scans were performed from 10° to 80° of 2-theta, with a step size of 0.03° and 4 sec per step. All samples were dried to thick films before the XRD analysis. The crystallinity index (CrI) of the samples was quantified using the empirical technique (Segal, Creely, Martin, & Conrad, 1959):

$$CrI = \frac{{I}_{002} - {I}_{am}}{{I}_{002}} \times 100 \qquad ( Eq . 1 )$$

where I₀₀₂ is the area of the crystalline region of the CNC and Iₐₘ is the area of the amorphous region.

### Results and Discussion

FTIR

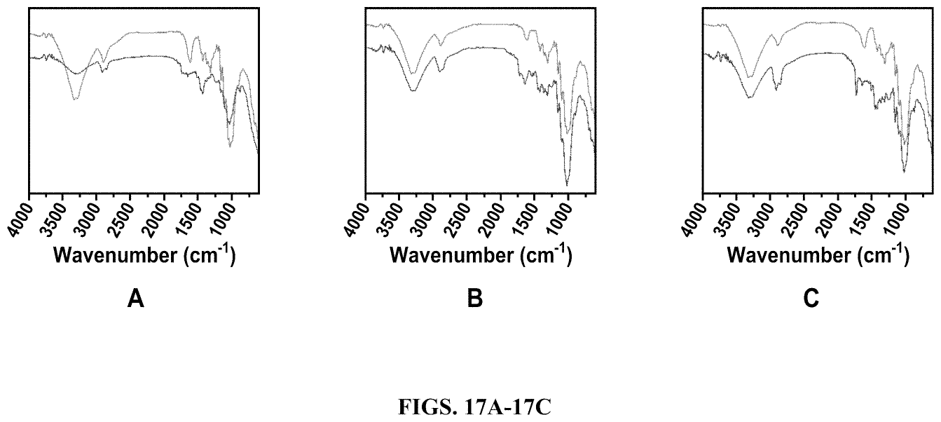

FIGS. 17A-17C show the FTIR spectra of the raw WCC, WPT, and WPTR, as well as the CNCs extracted from each feedstock. The peak at 1735 cm⁻¹ in the feedstock spectrum was caused by C═O stretching of hemicellulose's acetyl and uronic ester groups or ester linkage of lignin's ferulic and p-coumaric acids' carboxylic groups, which lost its intensity in the spectra of the extracted CNCs from all three feedstocks. This shows that the alkali treatment and bleaching were able to remove a high amount of lignin and hemicellulose. The bands at around 1509 cm⁻¹ in the feedstock spectrum were resulted by C═O in-plane aromatic vibrations, which were related to lignin (Sain & Panthapulakkal, 2006; X. F. Sun, Xu, Sun, Fowler, & Baird, 2005). The band at 1220 cm⁻¹ was attributed to lignin's ether linkages of stretching C—O—C, indicating that lignin was present in the feedstocks (Kumar, Negi, Choudhary, & Bhardwaj, 2014). Both the C—C ring breathing band and the C—O—C glycoside ether band, at 1155 cm⁻¹ and 1105 cm⁻¹, came from the cellulose of the three feedstocks (Garside & Wyeth, 2003). The antisymmetric bridge stretching vibration of the C—O group contributed to the peak at 1052 cm⁻¹ (Alemdar & Sain, 2008).

The bands of cellulose's functional groups were found to be consistent with those of the literature. The band at 2898 cm⁻¹, which was observed in all the samples, was a C—H stretching vibration of the total organic matter. The stretching vibrations of —OH group occurred at around 3330 cm⁻¹. The bending mode of absorbed water in cellulose was expected to be the cause of the sharp peak at 1640 cm⁻¹ in all the samples (S. M. L. Rosa, Rehman, de Miranda, Nachtigall, & Bica, 2012). At 1426 cm⁻¹, the presence of CH2 group in cellulose bends was observed. The signal at 1367 cm⁻¹ indicated that O—H bending vibrations existing (Trilokesh & Uppuluri, 2019). The sugar molecules' glycosidic bonds resulted in the peak at 898 cm⁻¹ (Haafiz, Hassan, Zakaria, & Inuwa, 2014).

The intensity difference between each sample is due to the difference of film thickness and variation in the concentration of the functional group associated with the molecular bond in each sample (Khairuddin et al., 2016).

TEM

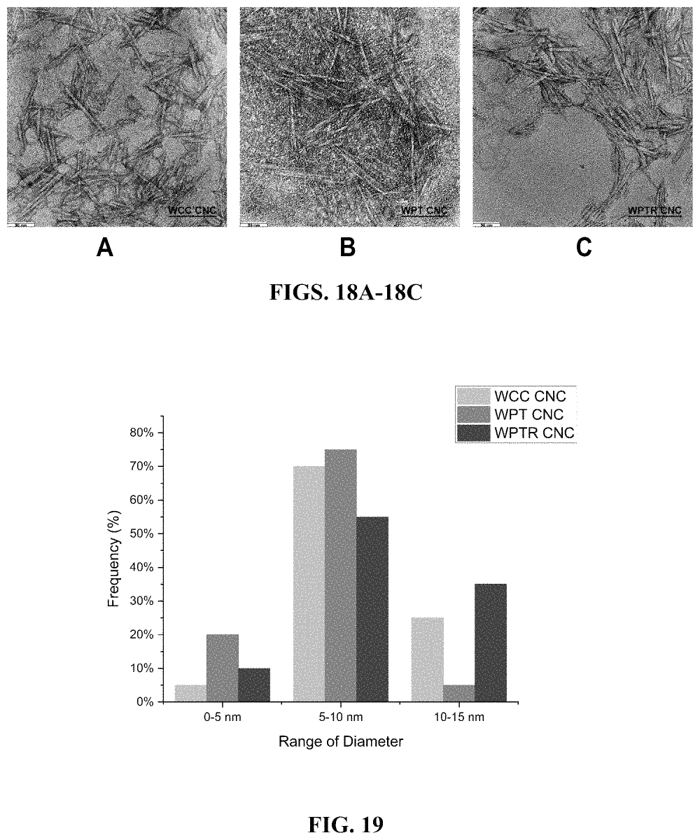

FIGS. 18A-18C show the TEM graphs of the CNCs extracted from WCC, WPT, and WPTR. The acid hydrolysis was predicted to cleave the amorphous area of cellulose microfibrils transversely while leaving the straight crystalline domains intact under controlled conditions. The procedure finally shrank the size of the fibers from micron to nanometer scales. The presence of nanoparticles in TEM images confirmed that the extraction was successful. These images depicted individual nanocrystals as well as certain aggregates. Because of the large specific area and strong hydrogen bonds formed between the nanocrystals, the appearance of laterally aggregated elementary crystallites in TEM images was expected. FIG. 19 depicts the distribution of the diameter of CNCs extracted from the three feedstocks using numerous TEM images. The diameter of the extracted CNCs is presented in Table 1.

TABLE 1
Diameter of the obtained CNC

| Sample | Diameter (nm) |
|---|---|
| Waste Corrugated Cardboards (WCC) | 8.77 ± 1.59 |
| Wastepaper Towels (WPT) | 6.32 ± 1.6 |
| Wastepaper Towel Cardboard Rolls (WPTR) | 8.32 ± 2.58 |

The diameter of the resulting CNCs from all three feedstocks ranged from 4 to 15 nm. The CNCs derived from all three feedstocks had similar physical properties as they were mostly derived from wood pulps. WPT CNCs were more whisker-shaped, whereas the two other samples were needle-like particles. WPT CNCs had a slightly smaller diameters when compared with WCC and WPTR CNCs. Smaller dimension for WPT CNCs was expected. Due to less lignin and hemicellulose content in WPT, cellulose fibers were highly affected by the pretreatment process, leading to more size reduction in comparison with the two other samples. This was also shown in SEM images that after the pretreatment process, WPT cellulose fibers were smaller in size.

XRD

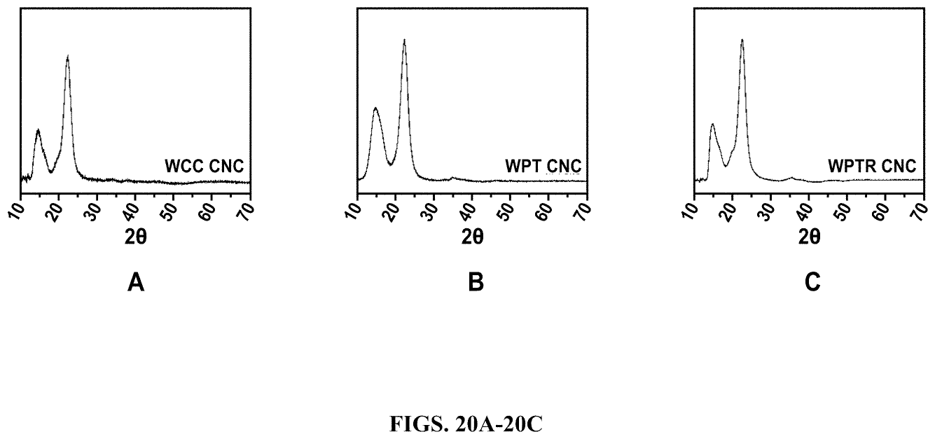

In contrast to hemicellulose and lignin, which are amorphous in nature, cellulose has a crystalline structure. The crystalline structure of cellulose is attributed to the hydrogen bonding interactions and Van der Waals forces between neighboring molecules. Acid hydrolysis has no impact on the crystalline domains but damages the fiber's amorphous area (Fengel and Wegener, 1984). The XRD peaks at around 2θ=15°, 22°, and 35° belongs to the crystalline characteristics of Cellulose. The XRD analysis of the extracted CNCs from WCC, WPT, and WPTR CNC are showed in FIGS. 20A-20C.

The XRD diagrams of the CNC extracted from the three feedstocks revealed similar peaks at around 2θ=16.0°, 22°, and a very slight one at 35°. The absence of a doublet signal at the peak around 22° suggested that the extracted cellulose was of type I. Table 2 shows that WPT CNCs have a slightly higher crystallinity index. Table 3 summarizes the crystallinity index (CrI) of the CNCs.

TABLE 2
The crystallinity index (Crl) of the
obtained CNC from WCC, WPT, and WPTR

| Sample | Crl (%) |
|---|---|
| Waste Corrugated Cardboards (WCC) | 78.1 |
| Wastepaper Towels (WPT) | 83.3 |
| Wastepaper Towel Cardboard Rolls (WPTR) | 77.5 |

TABLE 3
The crystallinity index (Crl) of the
obtained CNC from reported literature

| | Method of | Crl | |
|---|---|---|---|
| Source | Extraction | (%) | Reference |
| Soy hulls | Acid hydrolysis | 73.5 | (Flauzino Neto, |
| | | | Silvério, Dantas, |
| | | | & Pasquini, 2013) |
| Calotropis procera | Acid hydrolysis | 68.7 | (Song et al., 2019) |
| Pine | Acid hydrolysis | 56 | (Zhao, Zhao, & |
| | | | Yang, 2015) |
| Wastepaper | Acid hydrolysis | 75.9 | (Danial et al., 2015) |
| Ampelodesmos | Enzymatic | 86 | (F. Luzi et al., 2019) |
| mauritanicus | | | |
| Corn husk | Acid hydrolysis | 70.7 | (Zhao et al., 2015) |
| Rice husk | Acid hydrolysis | 59 | (Johar et al., 2012) |
| Pine needles | Bio-Enzyme | 78.46 | (Tang et al., 2021) |
| Borer powder of | Acid hydrolysis | 69.84 | (Hu et al., 2014) |
| bamboo | | | |
| Bagasse fibers | Acid hydrolysis | 77 | (Oliveira, Bras, |
| and pith | | | Pimenta, Curvelo, |
| | | | & Belgacem, 2016) |
| Maize straw | | 75.5 | |
| Pennisetum sinese | Acid hydrolysis | 77.3 | (Lu et al., 2014) |
| Pseudo-stem residue | Acid hydrolysis | 74 | (Meng et al., 2019) |
| Onion Skin | Acid hydrolysis | 30 | (Rhim et al., 2015) |
| Apple pomace | Acid hydrolysis | 78 | (Meliko\check{g}lu, Bilek, |
| | | | & Cesur, 2019) |
| Garlic straw | Acid hydrolysis | 68 | (Kallel et al., 2016) |
| Pseudostems of | Acid hydrolysis | 74 | (Mueller, Weder, & |
| banana plants | | | Foster, 2014) |
| Potato tuber | Acid hydrolysis | 68 | (Abe & Yano, 2009) |
| Sugarcane bagasse | Acid hydrolysis | 76 | (de Morais Teixeira |
| | | | et al., 2011) |
| Maize straw | Acid hydrolysis | 75.5 | (Rehman et al., 2014) |

It can be concluded that due to higher lignin and hemicellulose content in WCC and WPTR, they did not get removed completely after the pretreatment process and they affect the crystallinity of CNC, whereas the presence of higher cellulose content and less hemicellulose and lignin in WPT made it more susceptible to hydrolysis (Daicho, Saito, Fujisawa, & Isogai, 2018; Yoshida et al., 2008).

The CrI of the produced CNCs was close to that of the CNC isolated from wood described in the literature (Abe & Yano, 2009). The CrI of the CNC extracted from wood was reported to be 71%.

### Conclusion

The TEM observations indicate the presence of nano cellulose particles. The FTIR analysis showed the presence of cellulose and that the removal of lignin and hemicellulose from all three feedstocks was successful. The XRD analysis showed the high crystallinity index for the extracted CNCs. Among all three feedstocks, CNCs extracted from WPT had smaller diameter and higher crystallinity. This paper proposes a new path to household waste recycle process, by turning the waste into CNCs which is a valuable material. Finding the optimum hydrolysis time and temperature for all three feedstocks helped to reduce the use of harmful acid and make the process more environmentally friendly.

Other advantages which are obvious and which are inherent to the invention will be evident to one skilled in the art. It will be understood that certain features and sub-combinations are of utility and may be employed without reference to other features and sub-combinations. This is contemplated by and is within the scope of the claims. Since many possible embodiments may be made of the invention without departing from the scope thereof, it is to be understood that all matter herein set forth or shown in the accompanying drawings is to be interpreted as illustrative and not in a limiting sense.

### REFERENCES

Abe, K., & Yano, H. (2009). Comparison of the characteristics of cellulose microfibril aggregates of wood, rice straw and potato tuber. Cellulose, 16(6), 1017-1023.
Alemdar, A., & Sain, M. (2008). Isolation and characterization of nanofibers from agricultural residues: wheat straw and soy hulls. Bioresour Technol, 99(6), 1664-1671.
Bajpai, P. (2014). 7—Chemicals Used in Deinking and Their Function**Some excerpts taken from Bajpai, 2006 with kind permission from Pira International, UK. In P. Bajpai (Ed.), Recycling and Deinking of Recovered Paper (pp. 121-137). Oxford: Elsevier
Bjärestrand, A., & Alfthan, J. (2020). Improved properties of recycled fibre packages.
Chirayil, C. J., Joy, J., Mathew, L., Mozetic, M., Koetz, J., & Thomas, S. (2014). Isolation and characterization of cellulose nanofibrils from Helicteres isora plant. Industrial Crops and Products, 59, 27-34.
Daicho, K., Saito, T., Fujisawa, S., & Isogai, A. (2018). The Crystallinity of Nanocellulose: Dispersion-Induced Disordering of the Grain Boundary in Biologically Structured Cellulose. ACS Applied Nano Materials, 1(10), 5774-5785.
Dutta, S., Yu, I. K. M., Tsang, D. C. W., Fan, J., Clark, J. H., Jiang, Z., . . . Poon, C. S. (2020). Efficient Depolymerization of Cellulosic Paper Towel Waste Using Organic Carbonate Solvents. ACS Sustainable Chemistry & Engineering, 8(34), 13100-13110.
El Achaby, M., Kassab, Z., Aboulkas, A., Gaillard, C., & Barakat, A. (2018). Reuse of red algae waste for the production of cellulose nanocrystals and its application in polymer nanocomposites. International Journal of Biological Macromolecules, 106, 681-691.
EPA, U. S. E. P. A. Nondurable Goods: Product-Specific Data. (2021). https://www.epa.gov/facts-and-figures-about-materials-waste-and-recycling/nondurable-goods-product-specific-data Accessed.
EPA, U. S. E. P. A. Paper and Paperboard: Material-Specific Data. (2022). https://www.epa.gov/facts-and-figures-about-materials-waste-and-recycling/paper-and-paperboard-material-specific-data #:˜:text=Paper %20containers %20and %20packaging %2C %20excluding, 2018%20w as %204.2%20million %20tons. Accessed.
Fernandes, E. M., Pires, R. A., Mano, J. F., & Reis, R. L. (2013). Bionanocomposites from lignocellulosic resources: Properties, applications and future trends for their use in the biomedical field. Progress in Polymer Science, 38(10), 1415-1441.
Garside, P., & Wyeth, P. (2003). Identification of Cellulosic Fibres by FTIR Spectroscopy—Thread and Single Fibre Analysis by Attenuated Total Reflectance. Studies in Conservation, 48(4), 269-275.
Haafiz, M. K., Hassan, A., Zakaria, Z., & Inuwa, I. M. (2014). Isolation and characterization of cellulose nanowhiskers from oil palm biomass microcrystalline cellulose. Carbohydr Polym, 103, 119-125.
Habibi, Y., Lucia, L. A., & Rojas, O. J. (2010). Cellulose Nanocrystals: Chemistry, Self-Assembly, and Applications. Chemical Reviews, 110(6), 3479-3500.
Jiang, C., & Ma, J. (2000). De-inking of waste paper: flotation. Enzymatic Deinking Technologies, 1-2.
Johar, N., Ahmad, I., & Dufresne, A. (2012). Extraction, preparation and characterization of cellulose fibres and nanocrystals from rice husk. Industrial Crops and Products, 37(1), 93-99.
Kaushik, A., Singh, M., & Verma, G. (2010). Green nanocomposites based on thermoplastic starch and steam exploded cellulose nanofibrils from wheat straw. Carbohydrate Polymers, 82(2), 337-345.
Khairuddin, Pramono, E., Utomo, S. B., Wulandari, V., Zahrotul W, A., & Clegg, F. (2016). FTIR studies on the effect of concentration of polyethylene glycol on polimerization of Shellac. Journal of Physics: Conference Series, 776(1), 012053.
Kumar, A., Negi, Y. S., Choudhary, V., & Bhardwaj, N. K. (2014). Characterization of Cellulose Nanocrystals Produced by Acid-Hydrolysis from Sugarcane Bagasse as Agro-Waste. Journal of Materials Physics and Chemistry, 2(1), 1-8.
Mariano, M., Cercená, R., & Soldi, V. (2016). Thermal characterization of cellulose nanocrystals isolated from sisal fibers using acid hydrolysis. Industrial Crops and Products, 94, 454-462.
Mohanty, A. K., Misra, M., & Drzal, L. T. (2002). Journal of Polymers and the Environment, 10 (1/2), 19-26.
Ng, H. -M., Sin, L. T., Tee, T. -T., Bee, S. -T., Hui, D., Low, C. -Y., & Rahmat, A. R. (2015). Extraction of cellulose nanocrystals from plant sources for application as reinforcing agent in polymers. Composites Part B: Engineering, 75, 176-200.
Pandi, N., Sonawane, S. H., & Anand Kishore, K. (2021). Synthesis of cellulose nanocrystals (CNCs) from cotton using ultrasound-assisted acid hydrolysis. Ultrasonics Sonochemistry, 70, 105353.
Rosa, M. F., Medeiros, E. S., Malmonge, J. A., Gregorski, K. S., Wood, D. F., Mattoso, L. H. C., . . . Imam, S. H. (2010). Cellulose nanowhiskers from coconut husk fibers: Effect of preparation conditions on their thermal and morphological behavior. Carbohydrate Polymers, 81(1), 83-92.
Rosa, S. M. L., Rehman, N., de Miranda, M. I. G., Nachtigall, S. M. B., & Bica, C. I. D. (2012). Chlorine-free extraction of cellulose from rice husk and whisker isolation. Carbohydrate Polymers, 87(2), 1131-1138.
Sacui, I. A., Nieuwendaal, R. C., Burnett, D. J., Stranick, S. J., Jorfi, M., Weder, C., . . . Gilman, J. W. (2014). Comparison of the Properties of Cellulose Nanocrystals and Cellulose Nanofibrils Isolated from Bacteria, Tunicate, and Wood Processed Using Acid, Enzymatic, Mechanical, and Oxidative Methods. ACS Applied Materials & Interfaces, 6(9), 6127-6138.
Sain, M., & Panthapulakkal, S. (2006). Bioprocess preparation of wheat straw fibers and their characterization. Industrial Crops and Products, 23(1), 1-8.
Sasaki, M., Adschiri, T., & Arai, K. (2003). Production of Cellulose II from Native Cellulose by Near- and Supercritical Water Solubilization. Journal of Agricultural and Food Chemistry, 51(18), 5376-5381.
Segal, L., Creely, J. J., Martin, A. E., & Conrad, C. M. (1959). An Empirical Method for Estimating the Degree of Crystallinity of Native Cellulose Using the X-Ray Diffractometer. Textile Research Journal, 29(10), 786-794.
Sheltami, R. M., Abdullah, I., Ahmad, I., Dufresne, A., & Kargarzadeh, H. (2012). Extraction of cellulose nanocrystals from mengkuang leaves (Pandanus tectorius). Carbohydrate Polymers, 88(2), 772-779.
Sun, N., Rodriguez, H., Rahman, M., & Rogers, R. D. (2011). Where are ionic liquid strategies most suited in the pursuit of chemicals and energy from lignocellulosic biomass? Chem. Commun., 47(5), 1405-1421.
Sun, X. F., Xu, F., Sun, R. C., Fowler, P., & Baird, M. S. (2005). Characteristics of degraded cellulose obtained from steam-exploded wheat straw. Carbohydr Res, 340(1), 97-106.
Trilokesh, C., & Uppuluri, K. B. (2019). Isolation and characterization of cellulose nanocrystals from jackfruit peel. Scientific Reports, 9(1), 16709.
Wei, H., Rodriguez, K., Renneckar, S., & Vikesland, P. J. (2014). Environmental science and engineering applications of nanocellulose-based nanocomposites. Environ. Sci.: Nano, 1(4), 302-316.

What is claimed is:

1. A method of making a plant growth medium, comprising isolating cellulose nanocrystals from a feedstock; and adding the cellulose nanocrystals to the plant growth medium.

2. The method of claim 1, wherein isolating cellulose nanocrystals comprises extracting cellulose from the feedstock and extracting the cellulose nanocrystals from the cellulose.

3. The method of claim 2, wherein extracting cellulose from the feedstock comprises acidic or enzymatic hydrolysis, cryocrushing the feedstock, high shear grinding, or any combination thereof.

4. The method of claim 3, wherein the enzymatic hydrolysis uses cellulolytic enzymes from Trichoderma reesei.

5. The method of claim 2, wherein extracting cellulose nanocrystals from the cellulose comprises acidic or enzymatic hydrolysis, performing ultrasonication on the cellulose, performing high energy bead milling on the cellulose, or any combination thereof.

6. The method of claim 1, wherein the cellulose nanocrystals are rod-shaped, spherical, or any combination thereof.

7. The method of claim 1, wherein the cellulose nanocrystals are not comprised of lignin or hemicellulose.

8. The method of claim 1, wherein the cellulose nanocrystals have a width from 3 nm to 100 nm.

9. The method of claim 1, wherein the cellulose nanocrystals have a length from 50 nm to 6 μm.

10. The method of claim 1, wherein the plant growth medium comprises a plant seed.

11. The method of claim 1, wherein the plant growth medium further comprises a germination material.

12. A plant starter plug comprising the plant growth medium made by the method of claim 11.

13. A plant growth medium comprising germination material and cellulose nanocrystals.

14. The plant growth medium of claim 13, further comprising a plant seed.

15. The plant growth medium of claim 13, wherein the cellulose nanocrystals are substantially free of amorphous cellulose.

16. The plant growth medium of claim 13, wherein the cellulose nanocrystals are rod-shaped, spherical, or any combination thereof.

17. The plant growth medium of claim 13, wherein the cellulose nanocrystals are not comprised of lignin or hemicellulose.

18. The plant growth medium of claim 13, wherein the cellulose nanocrystals have a width from 3 nm to 100 nm.

19. The plant growth medium of claim 13, wherein the cellulose nanocrystals have a length from 50 nm to 6 μm.

20. A plant starter plug comprising the plant growth medium of claim 13.
